(19) United States
(12) Patent Application Publication
(10) Pub. No.: US 2011/0280767 A1
(43) Pub. Date: Nov. 17, 2011

(54) DEVICE FOR DECONTAMINATION BY MISTING

(76) Inventor: Frederic Goessens, Vieillevigne (FR)

(21) Appl. No.: 13/145,696

(22) PCT Filed: Jan. 22, 2010
(86) PCT No.: PCT/FR2010/000057

§ 371 Date: Jul. 21, 2011

(30) Foreign Application Priority Data
Jan. 23, 2009 (FR) 0900302

(51) Int. Cl. A61L 2/18; G05D 9/00

(52) U.S. Cl. 422/106; 422/292; 422/119

(57) ABSTRACT

The present invention relates to a device for decontamination of a room by misting a liquid treatment product (3) into the volume thereof, of the type comprising a receiving tank (2) for said product (3), means (4) suited for generating fine droplets of the product (3) in a zone of the surface of the room, means (6) suited for forming an air flow capable of entraining the fine droplets by mixing therewith, means (10, 12) suited for injecting the mist thus formed into the room. This device is characterized in that the air flow traverses a convergent tube (18) whose axis (xx′) is coincident with that of the air flow, where this tube opens out near the surface of the liquid product (3) and is inclined at an angle (β) relative thereto.

The present invention relates to a device for decontamination by misting a disinfecting product onto the surfaces to be treated and in particular the walls and objects in a room.

It is known that bacterial agents which are the cause of contamination and which are suspended in the air in a room have a tendency to settle onto the various surfaces and objects contained therein. It is further known that inversely bacterial agents which develop on the objects and walls in a room (for example operating rooms, clean rooms or various care rooms, etc.) have a tendency to go into suspension in the atmosphere. These rooms are therefore found in a situation of continual exchange between the walls and objects on the one hand and the atmosphere on the other.

Ensuring the overall decontamination, meaning both the atmosphere and various walls and objects in a room, is proposed by spraying a disinfecting product into the volume thereof. It has been observed this kind of disinfection by spraying has several drawbacks.

First, the size of the droplets formed is relatively large (of order 80 to 200 μm for flow rate of 3 to 5 mL of air per minute), such that droplets deposit on the surfaces near the site of their spraying by simple force of gravity, which of course is not satisfactory to the extent where the surfaces far from the spraying injector go untreated.

Second, because of the large size of the droplets, they have a tendency to combine and form a damp film, even liquid pools, on the surfaces of the walls and objects in the MOM.

In the patent FR 2,859,650 in the name of the applicant, improving the fractionating of the spray droplets was proposed by making use of an injection device making it possible to obtain fine droplets whose dimensions are of order 2 μm to 20 μm and which thus have the property of being found in suspension in the entire volume of the room and depositing on the walls and objects contained therein without clumping together so well that they form a continuous film; the mist thus generated is called “dry mist”.

According to a variant of this invention, the injection device is provided with an ultrasonic “resonator” placed downstream from the injector outlet, such that the flow exiting therefrom finds itself subject to a fragmentation forming a sort of “diffraction” of the drops, having the effect of making them still smaller which makes it possible to further increase the homogeneity of their distribution.

In the application FR 09.000134 the applicant has also proposed a device for treatment by misting in which the fragmentation is obtained by making use of a specific injector comprising a main vein successively constituted of a convergent axial vein intended to receive a pressurized gas flow, a cylindrical vein, and a divergent axial vein, where said injector comprises furthermore at least one secondary vein, substantially transverse, intended to admit a flow of treatment liquid and which opens out downstream from the convergent vein, and where the axis of the cylindrical vein is offset in angle relative to the longitudinal axis of the convergent and divergent veins.

Such a device makes it possible in a room to be treated of large volume, of order 200 to 300 m³, to thereby create a “dry mist” constituted from the treatment product, making it possible to attain a total eradication of any bacterial germ.

From the patent US 2008/223,953 a device is known for spraying a treatment product making use of piezoelectric means for generating on the surface thereof fine droplets which are next entrained in a flow of air. However it will be noted that because of the constitution thereof, the airflow generated by this device undergoes multiple reflections before reaching the surface of the liquid which has the effect of reducing the flow speed and increasing the noise generated by the device and does so to the detriment of the diffusion of the treatment liquid.

The purpose of the present invention is to propose a treatment device of the same type, in particular intended for treatment of small volume rooms, of order 50 m³. By generating a flow of air arriving on the surface of the treatment liquid at a significant speed, this device makes the entrainment of the liquid microparticles released by the piezoelectric means and their misting into the atmosphere easier, and does so with non-complex airflow generation means such as for example a simple fan.

The device according to the invention is therefore low weight and low volume which makes it easily transportable and easy and quick to set up, in particular without requiring an electric connection to an electric outlet.

Thus the object of the present invention is a device for decontamination of a room by misting a liquid treatment product into the volume thereof, of the type comprising a tank for receiving said product, means suited for generating fine droplets of product in a zone of the surface thereof, means suited for forming an airflow able to entrain these fine droplets by mixing them therewith, and means suited for injecting into the room the mist thus formed, characterized in that the airflow crosses a convergent tube whose axis is coincident with that of said airflow, where this tube opens out near the surface of the liquid product and is inclined relative thereto.

The angle of inclination of the convergent tube will be included between 20 and 60° and will be preferably of order 45° relative to the surface of the liquid.

The means for formation of the airflow could be constituted by a fan and the means for generation of the droplets could be constituted of a piezoelectric generator placed in the bottom of the tank.

Preferably the airflow leaving the convergent tube will be directed onto the surface of the treatment liquid located above the piezoelectric generator.

The decontamination device according to the invention could comprise means for controlling the size of the treatment liquid droplets mixed with the airflow. These control means could be constituted of a substantially vertical cylindrical tube with a set height, an upstream end of which will be placed above the treatment liquid droplet generation zone and the downstream end will be provided with an injection nozzle in particular constituted of a convergent line.

The tank could comprise treatment liquid supply means and also high and low level sensors for the liquid in the tank which are associated with level control means. Further, the tank could also comprise at least one sensor for detecting the absence of liquid.

The various sensors with which the device is equipped could be provided with elements for protection against disturbances brought by the transducer.

The means of supply could be constituted of a pump, for example a reversible pump, in particular peristaltic type.

The decontamination device could be provided with electronic means for calculation of the flow rate of the means of supply of treatment liquid based on the measurement of the time necessary for said means of supply to bring the liquid level from one sensor to another sensor. It could also comprise electronic means for calculation of the distribution rate thereof by the measurement of the time necessary for the device to lower the level of liquid to be distributed from a detection position of an upper sensor to a detection position of a lower sensor.

Finally, the device according to the invention could comprise means suited, especially at end of cycle, for recovering by means of said pump the treatment liquid remaining in the tank for returning it into a storage barrel.

As a nonlimiting example, an embodiment of the present invention will be described below with reference to the attached drawing in which:

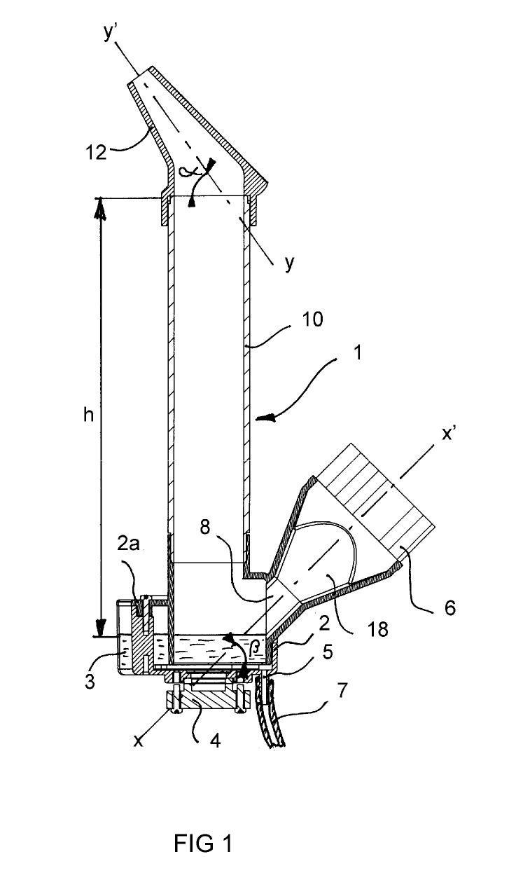

FIG. 1 is a vertical and transverse section view of a decontamination device according to the invention.

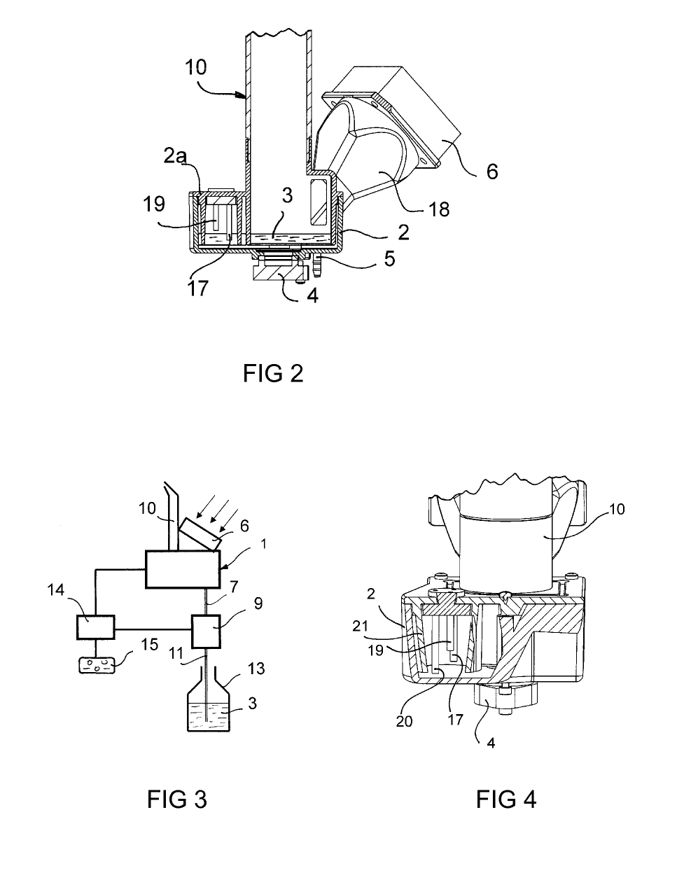

FIG. 2 is a partial vertical section view of the decontamination device shown in FIG. 1.

FIG. 3 is a representation in schematic form of the various circuits entering into the constitution of the decontamination device according to the invention.

FIG. 4 is a partially exploded perspective view of an implementation variant of the tank for the device according to the invention.

FIG. 1 shows an implementation example of a decontamination device 1 according to the invention.

In this figure the device includes a tank 2 intended to receive a treatment liquid 3 that should be distributed in dry mist form in the room to be treated. This tank 2 is closed by a lid 2a which preferably makes it possible to seal it and is provided at the base thereof with a piezoelectric ceramic transducer 4 which, in a well-known manner, is suited to vibrating when it is powered at the resonant frequency thereof by a high-frequency generator not shown in the drawing, in particular of order 1.8 MHz.

In the present embodiment of the invention the means for generation of the airflow are constituted of a fan 6 followed by a convergent tube 18 and a cylindrical line 8. The same 6 is mounted such that, when it is operating, it draws air from the outside and propels it through the lines 18 and 8 in the form of a flow of order 30 L/min.

The axis xx′ along which the fan 6 propels the airflow is coincident with the axis of the convergent tube 18 and the axis of the cylindrical line 8. This common axis xx′ is inclined at an angle βrelative to the surface of the liquid with a value included between 20° and 60° and is preferably near 45° so as to be directed onto the zone of the surface of the liquid 3 which is located above the transducer 4 and which is therefore excited thereby.

The bottom of the tank 2 is provided with a tip 5 making it possible, as shown in FIG. 3, to connect it through a flexible tube 7 to a supply pump 9, for example a reversible peristaltic pump, which is connected by a line 11 to a reservoir, or cartridge 13, containing the treatment product. The device includes electronic control means, in particular constituted of a microcontroller 14 which is interfaced to a control console 15 and which is connected to the pump 9, the fan 6 and also to the means for control of the level of the liquid present in the tank 2.

Above the transducer 4 is placed a vertical tube 10 which is terminated at the upper portion thereof by a convergent nozzle 12 whose axis yy′ is inclined at an angle α relative to the surface of the liquid or the bottom of the tank 2. Preferably this angle α is of order 45°.

Under these conditions, operation of the device according to the invention is set up as follows.

When the transducer 4 is excited by powering it with a current at a frequency preferably close to its resonant frequency, it enters into vibration and creates within the treatment liquid a cavitation effect generating gaseous bubbles which burst after rising to the surface thereby forming a fine mist. The effect of the airflow generated by the fan 6 is to recover this mist and to entrain it mixed with the airflow to the outside through the tube 10.

The acceleration in the tube 18 of the airflow generated by the fan 6 makes it possible to improve the effectiveness of the entrainment of such a mixture and thereby have an entrainment power reserve which will make it possible to modulate, as described below, the size of the entrained drops of liquid.

In fact it has been noted that a mist thus formed comprises both microdroplets and larger droplets. It is therefore necessary to eliminate these latter which would have, if they were retained, a wetting effect which should be avoided for this type of application. The vertical tube 10 has in particular a function of controlling and limiting the size of the drops sprayed out of the device. It has also been observed that by adjusting the power of the air flow blown by the fan 6 and also the height h of the tube 10, the dimension of the drops at the outlet could be controlled to the extent where the larger of these drops fall back into the tank 2 under the force of their own weight. In other words, the designer of the device according to the invention has the possibility of controlling the size of the drops at the outlet of the device by acting on the power of the fan 6 and on the height h of the tube 10.

Further it was observed that by giving the outlet nozzle 12 placed in the extension of the tube 10 a convergent shape the quality of the outlet jet was improved by avoiding vortices which made it possible to form a denser mist.

The level control means, as shown in FIG. 2, include two sensors, specifically a low level detection sensor 17 and a high level detection sensor 19 which start from the lid 2a of the tank 2 and which extend vertically downwards towards the bottom thereof to stop at a distance therefrom respectively equal to the low level and the high level. The sensors 17 and 19 are connected to the microcontroller 14 which orders filling of the tank 2 when the treatment liquid level falls below the low level and stops filling when the liquid reaches the high level. In the present embodiment of the invention, high and low level detectors are placed such that the height of the water above the transducer 4 remains included between 15 mm and 21 mm.

In a particularly interesting variant of the present invention, shown in FIG. 4, the device comprises a third sensor 22 placed near the bottom of the tank 2 such that is suited for detecting complete absence of liquid therein. It is designated below as zero level sensor.

The three sensors are associated with electronic management means, for example the microcontroller 14, which in particular make it possible both to determine at the beginning of the cycle the actual flow rate ρ1 of the pump 9 and, also to measure during operation of the device the diffusion rate ρ2, meaning the flow rate of the treatment liquid actually diffused into the room.

To do this, the microcontroller 14 measures at the beginning of the cycle the time t1 necessary for the pump 9 to fill the volume V1 of the tank 2 included between the low and high levels with liquid. The actual flow rate ρ1 of the pump 9 is thus: /ρ1=V1/t1.

Furthermore, the user by means of the control console 15 has the possibility of entering the volume Vd that they want to mist into the room during the treatment cycle, such that the microcontroller 14 is then able to calculate the operating time T of the pump 9 necessary for it to bring into tank 2 this volume Vd of treatment product, which is T=Vd/ρ1. Once the time T has elapsed, the microcontroller 14 orders the final stoppage of the pump 9 for this treatment cycle and the misting continues until the zero level sensor detects the complete emptying of the tank 2 which marks the end of the treatment cycle.

During a treatment cycle, it is understood that the microcontroller 14 orders the interruption of the operation of the pump 9 once the liquid reaches the high sensor 19 and then orders the resumption thereof once the low sensors 17 is no longer submerged. The overall operating time T is therefore constituted of the sum of different operating times of the pump between the high and low levels.

Additionally, during operation the microcontroller 14 is able to compute the time T2 which elapses between the instant when the liquid is at the high level (detection by sensor 17) and the time when it is at the low level (detection by sensor 19), which corresponds to the misting of a volume V2 of liquid equal to that included between the two sensors. It is then able to determine the diffusion rate p2 which is the flow rate of the liquid used, ρ2=V2/t2. If this proves to be insufficient, an appropriate programming of the microcontroller then makes it possible for the microcontroller to act in order to increase the flow rate for example by increasing the supply voltage to the piezoelectric transducer 4.

In order to avoid the sensors being bothered by the disruptions generated by the vibrations of the transducer 4, they can, as shown in FIG. 4, be protected by partitions 21 which surround them without bothering the access to the treatment liquid.

Preferably the tanks 2 will be sealed which will make it possible for the user to move the device easily without risk of leaks to the outside thereof.

Furthermore, to the extent where the pump 9 is reversible type, at the end of the cycle the microcontroller can order the recovery of the treatment liquid remaining in the tank 2, meaning the liquid remaining between the low level sensors 17 and zero level sensor in order to bring it back into the cartridge 13.

What is claimed is:

1. A device for decontamination of a room by misting a liquid treatment product (3) into the volume thereof, of the type comprising a receiving tank (2) for said product, means (4) suited for generating fine droplets of product (3) in a zone of the surface thereof, means (6) suited for forming an airflow able to entrain these fine droplets by mixing them therewith, and means (10, 12) suited for injecting into the room the mist thus formed, characterized in that the airflow crosses a convergent tube (18) whose axis (xx′) is coincident with that of said airflow, where this tube opens out near the surface of the liquid product (3) and is inclined at an angle (β) relative thereto.

2. Decontamination device according to claim 1 characterized in that the angle of inclination (5) of the convergent tube (18) will be near 45° relative to the surface of the liquid.

3. Decontamination device according to one of claims 1 or 2, characterized in that the means for formation of the airflow are constituted by a fan (6).

4. Decontamination device according to one of the preceding claims, characterized in that the means for generation of the droplets are constituted of a piezoelectric generator (4).

5. Decontamination device according to claim 4 characterized in that the piezoelectric generator (4) is placed in the bottom of the tank (2).

6. Decontamination device according to one of claims 4 or 5, characterized in that the airflow leaving the convergent tube (18) is directed onto the surface of the treatment liquid located above the piezoelectric generator (4).

7. Decontamination device according to one of the preceding claims, characterized in that it comprises means for controlling (10) the size of the treatment liquid droplets mixed with the airflow.

8. Decontamination device according to claim 7 characterized in that said control means are constituted of a substantially vertical cylindrical tube (10) with a set height (h), an upstream end of which is placed above the treatment liquid droplet generation zone and the downstream end is provided with an injection nozzle (12).

9. Decontamination device according to claim 8 characterized in that injection nozzle is constituted by a convergent line (12).

10. Decontamination device according to one of the preceding claims, characterized in that the tank (2) comprise treatment liquid (3) supply means (9) and also high (19) and low (17) level sensors for the liquid in the tank (14) which are associated with level control means.

11. Decontamination device according to claim 10 characterized in that the tank comprises a sensor (20) for detecting the absence of liquid.

12. Decontamination device according to claim 10 characterized in that the sensors (17, 19, 20) are provided with elements for protection (21) against disturbances caused by the means suited to generate fine droplets.

13. Decontamination device according to one of claims 10 to 12, characterized in that the means of supply are constituted of a pump (9), for example a reversible pump, in particular peristaltic type.

14. Decontamination device according to one of claims 10 to 13, characterized in that it comprises electronic means for calculation (14) of the flow rate (ρ1) of the means of supply (9) of treatment liquid based on the measurement of the time (t1) necessary for said means of supply to bring the liquid level from one sensor to another sensor.

15. Decontamination device according to one of claims 10 to 14, characterized in that it comprises electronic means for calculation (14) of the distribution rate thereof (ρ2) by the measurement of the time (t2) necessary for the device to lower the level of liquid to be distributed from a detection position of an upper sensor to a detection position of a lower sensor.

16. Decontamination device according to one of claims 13 to 15, characterized in that comprise means suited, especially at end of cycle, for recovering by means of said pump (9) the treatment liquid remaining in the tank (2) for returning it into a storage barrel.
